(19) United States
(12) Patent Application Publication
(10) Pub. No.: US 2008/0097397 A1
(43) Pub. Date: Apr. 24, 2008

(54) Vascular introducer sheath

(76) Inventor: Anthony C. Vrba, Maple Grove, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/449,098

(22) Filed: Jun. 8, 2006

(51) Int. Cl. A61M 25/08

(52) U.S. Cl. 604/525

(57) ABSTRACT

An introducer sheath that can include an elongate tubular member, such as a metallic tubular member, including a tubular wall defining a lumen and including a proximal portion and a distal portion. In some embodiments, a portion of the tubular member, such as the distal portion, can include a plurality of apertures defined in the tubular wall, and another portion, such as the proximal portion, can be free of apertures defined in the tubular wall. The portion including the apertures defined therein can be more flexible than the portion free of the apertures. A second tubular member can be disposed on or within the elongate tubular member, and can define a fluid tight pathway through the lumen. Additionally, a hub can be attached to the proximal portion of the elongate tubular member and in fluid communication with the fluid tight pathway.

### FIELD OF THE INVENTION

The invention generally relates to introducer sheaths for use in procedures requiring vascular access. More specifically, the invention relates to introducer sheaths including an elongated shaft including a metallic sleeve including a portion including slots and/or apertures defined therein.

### BACKGROUND

Vascular introducer sheaths are well known components of vascular access systems which are used in a wide variety of diagnostic and therapeutic vascular procedures, such as angiography, angioplasty, thermolysis, and embolization procedures. Vascular access systems typically include an introducer sheath for use in combination with a guide wire and a dilator. The introducer sheaths usually include a hemostatic or hemostasis valve which inhibits blood loss as guide wires, catheters and the like are introduced and manipulated in the vasculature via the sheath.

A variety of vascular introducer sheaths have been developed over the past several decades. Because gaining access to the vascular anatomy of a patient may be a somewhat intricate procedure, it is desirable to combine a number of performance features into the introducer sheaths used. A number of different introducer sheaths structures and assemblies are known, each having certain advantages and disadvantages. However, there is an ongoing need to provide alternative introducer sheaths structures and assemblies.

### SUMMARY OF SOME EMBODIMENTS

The invention relates to alternative introducer sheath structures, assemblies, manufacturing methods, and methods of use. Some embodiments relate to an introducer sheath that can include an elongate tubular member, such as a metallic tubular member, including a tubular wall defining a lumen and including a proximal portion and a distal portion. In some embodiments, a portion of the tubular member, such as the distal portion, can include a plurality of apertures defined in the tubular wall, and another portion, such as the proximal portion, can be free of apertures defined in the tubular wall. The portion including the apertures defined therein can be more flexible than the portion free of the apertures. A second tubular member can be disposed on or within the elongate tubular member, and can define a fluid tight pathway through the lumen. Additionally, a hub can be attached to the proximal portion of the elongate tubular member and in fluid communication with the fluid tight pathway.

In some embodiments, the introducer sheath may include a relatively high level of pushability and torqueability, particularly near its proximal end, such that the sheath can be advanced through and into the anatomy as desired. The sheath may also be relatively laterally flexible, particularly near its distal end, such that the sheath can be adapted to enter the anatomy at a desired angle, and resist kinking. In some embodiments, the use of apertures defined in a tubular wall may provide for the desired degree of lateral flexibility in the distal portion, but may also allow the distal portion to maintain a desired degree of torqueability and/or pushability.

The above summary of some embodiments is not intended to describe each disclosed embodiment or every implementation of the present invention. The Figures, and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a partial side plan view of one example embodiment of an introducer sheath;

FIG. 2 a partial cross-sectional side view of the introducer sheath of FIG. 1; and

FIG. 3 is a partial cross sectional side view of the introducer sheath of FIG. 1 shown disposed within the anatomy of a patient.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF SOME EMBODIMENTS OF THE INVENTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

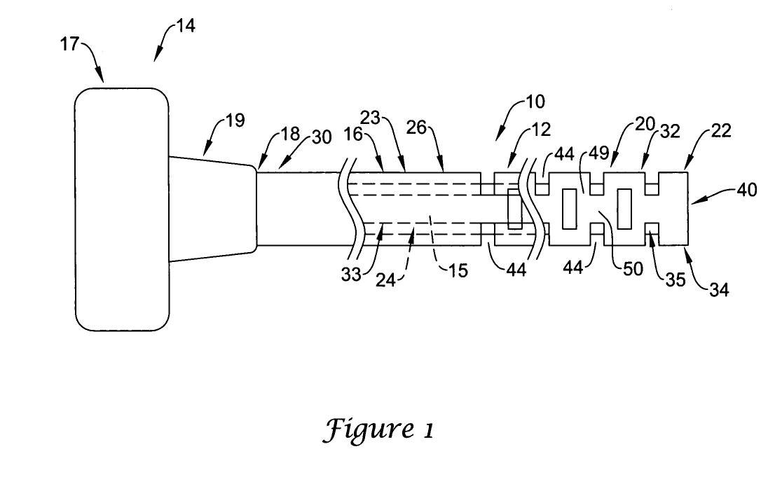

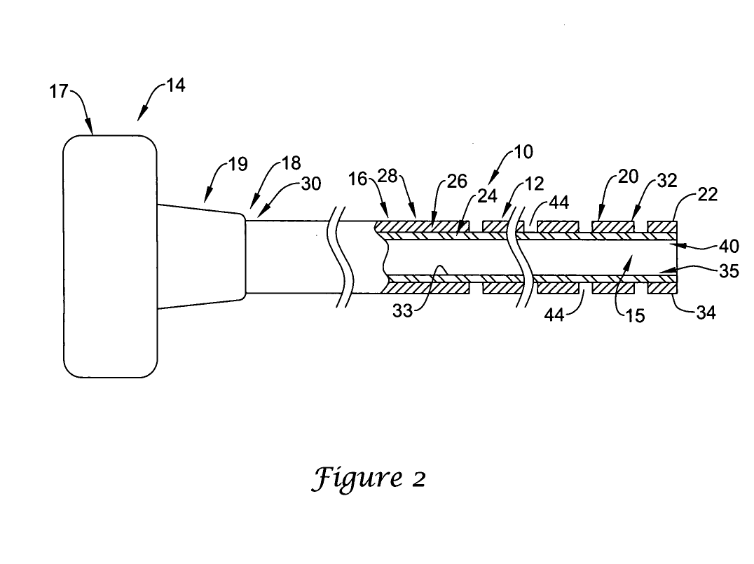

Refer now to FIGS. 1 and 2, which illustrate an introducer sheath 10 in accordance with one example embodiment. The introducer sheath 10 includes an elongate shaft 12 including a proximal portion 16 having a proximal end 18, and distal portion 20 having a distal end 22. The shaft 12 is a generally tubular construction defining a lumen 15 therein. A manifold and/or hub 14 can be connected to the proximal end 18 of the elongate shaft 12, and include a lumen and/or other structure to provide access and/or fluid communication to 15 lumen within the shaft 12, and/or to facilitate the insertion of and/or connection of other medical devices (e.g., guidewire, catheter, syringe, Y-adapter, etc.) within and/or to the shaft 12. The shaft 12 includes a multi-layer construction including a first tubular member 26 and a second tubular member 24. In the embodiment shown, the first tubular member 26 may be an outer tubular member, and the second tubular member 24 may be an inner tubular member, but in other embodiments, the two tubular member may be reversed such that the first tubular member 26 may be an inner tubular member, and the second tubular member 24 may be an outer tubular member.

The first tubular member 26 includes a proximal portion 28 having a proximal end 30, and distal portion 32 having a distal end 34. The proximal and distal portions 28/32 of the first tubular member 26 may generally correspond to the proximal and distal portions 16/20 of the shaft 12. The first tubular member 26 can include one or more portions that include a plurality of apertures 44 defined therein, as will be discussed further below.

The first tubular member 26 can be disposed about at least a portion of the second tubular member 24 at a location along the length of the shaft 12 between proximal end 18 and distal end 22. In the embodiment shown, the first tubular member 26 is disposed about the second tubular member 24 along substantially the entire length of the shaft 12, but in other embodiments, may only extend along a portion of the length of the shaft 12 and/or second tubular member 24. The length of the first tubular 26 can also vary, depending upon, for example, the length of the shaft 12, the desired characteristics and functions of the introducer sheath 10, and other such parameters. In some embodiments, the first tubular member 26 has a length that allows it to be disposed over the majority of the length of the second tubular member 24. In yet other embodiments, the first tubular member 26 may extend distally and/or proximally beyond the second tubular member 24. As an example, the shaft 12 may have a length of about 5 centimeters or more, in the range of about 5 to about 100 cm, in the range of about 10 to 100 cm, or in the range of about 12 to about 100 cm. The length of the first tubular member 26 can be about 5 centimeters or more, in the range of about 5 to about 100 cm, in the range of about 10 to 100 cm, in the range of about 12 to about 100 cm, or in the range of about 20 to about 100 cm.

The first tubular member 26 defines a lumen 40 that can be adapted and/or configured to house or surround a portion of the second tubular member 24. In this regard, the first tubular member 26 typically has an inner diameter that is about the same as or greater than the outer diameter of the second tubular member 24. As such, the first tubular member 26 can be disposed about the second tubular member 24, and/or a portion of the second tubular member 24 is disposed within the lumen 40 of the first tubular member 26. In some embodiments, the outer surface of the second tubular member 24 and the inner surface of the first tubular member 26 are in contact with each other such that there is no gap or space between them. However, in other embodiments, the outer surface of the second tubular member 24 and the inner surface of the first tubular member 26 are sized and/or shaped such that one or more gaps or spaces can be defined between them. Such a gap or space may remain open or unfilled by any other structure of the sheath, with the exception of small coupling points. However, in other embodiments, other structures of the sheath 10 or additional attachment points along the length of the first tubular member 26 may be used, and as a result, some portion of any such gaps may be filled by such structures. In some embodiments, the first tubular member 26 can have an inner diameter, defining the lumen 40, that is in the range of about 0.005 to about 0.50 inches in size, and in some embodiments, in the range of about 0.01 to about 0.30 inches in size, or in the range of about 0.05 to about 0.26 inches in size. Additionally, in some embodiments, the first tubular member 26 can have an outer diameter that is in the range of about 0.005 to about 0.75 inches in size, and in some embodiments, in the range of about 0.01 to about 0.30 inches in size, or in the range of about 0.05 to about 0.26 inches in size. It should be understood however, that these, and other dimensions provided herein, are by way of example embodiments only, and that in other embodiments, the size of the inner and outer diameter of the first tubular member 26 can vary greatly from the dimensions given, depending upon the desired characteristics and function of the device.

The first tubular member 26 can act to reinforce or impart desired properties, such as tortional and lateral rigidity, to the shaft 12, and as such can be adapted and/or configured to have a desired level of stiffness, torqueability, flexibility, and/or other characteristics. Those of skill in the art and others will recognize that the dimensions, structure, and materials of the first tubular member 26 are dictated primary by the desired characteristics, and the function of the final sheath 10, and that any of a broad range of the dimensions, structure, and materials can be used.

The desired stiffness, torquability, lateral flexibility, bendability or other such characteristics of the first tubular member 26 can be imparted or enhanced by the structure of the first tubular member 26. For example, as indicated above, the first tubular member 26 may include a thin wall tubular structure, including one or a plurality of apertures 44, such as grooves, cuts, slits, slots, or the like, formed along the entire length or a portion of the length of the first tubular member 26. For example, in the embodiment shown, the distal portion 32 can include a plurality of apertures 44 defined in the tubular wall of the first tubular member 26, and the proximal portion 28 can be free of apertures defined in the tubular wall. The presence of the apertures 44 within the distal portion 32, and the absence of such the apertures 44 within the proximal portion 28 may provide the shaft 12 with certain desirable characteristics. Such structure may be desirable because it may allow first tubular member 26, or portions thereof (e.g. the distal portion 32), to have a desired level of laterally flexibility as well as have the ability to transmit torque and pushing forces from the proximal portion 16 to the distal portion 20 of the shaft 12. For example, in some embodiments, the proximal portion 28 may include a relatively high level of pushability and torqueability, such that the sheath 10 can be advanced through and into the anatomy as desired. The distal portion 32, due to the presence of the apertures 44, may be relatively more laterally flexible than the proximal portion 28, such that the sheath 10 can be flexed, or otherwise adapted to enter the anatomy at a desired angle, and resist kinking. However, due to the distal portion 32 being a tubular structure including apertures 44 defined in a tubular wall, the distal portion 32 may still maintain a relatively high level of pushability and torqueability.

In some embodiments, the distal about 10% to about 90%, or the distal about 20% to about 80%, of the total length of the first tubular member 26, and/or the total length of the shaft 12, can include apertures 44 defined in the first tubular member 26. Likewise, the proximal about 10% to about 90%, or about 20% to about 80%, of the total length of the first tubular member 26, and/or the total length of the shaft 12, is free of such apertures 44. For example, in some embodiments, the distal portion 32 may extend along in the range of about 5% to about 98%, or in the range of about 10% to about 90%, or in the range of about 20% to about 80% of the total length of the first tubular member 26 and/or the total length of the shaft 12. Likewise, the proximal portion 28, which may be free of apertures 44, may extend along in the range of about 2% to about 90%, or in the range of about 10% to about 90%, or in the range of about 20% to about 80%, of the total length of the first tubular member 26 and/or the total length of the shaft 12.

As an example, in some embodiments, the distal portion 32 may have a length of about 5 cm or greater, in the range of about 5 to about 100 cm, or in the range of about 10 to about 100 cm, in the range of about 12 to about 100 cm, or in the range of about 20 to about 100 cm, and includes apertures 44 defined therein, and the proximal portion 32 may make up the remainder of the length of the first tubular member 26 and/or the shaft 12. Likewise, in some embodiments, the proximal portion 28 may have a length of about 2 cm or more, or in the range of 2 to about 40 cm, or in the range of about 4 to about 20 cm, and is free of apertures 44 defined therein, while the distal portion 28, including apertures 44 defined therein, may make up the remainder of the length of the first tubular member 26 and/or the shaft 12. It should be understood however, that these, and other dimensions provided herein, are by way of example embodiments only, and that in other embodiments, the disposition of apertures 44 can vary greatly from the dimensions given, depending upon the desired characteristics and function of the device.

The apertures 44 can be formed in essentially any known way. For example, apertures 44 can be formed by methods such as micro-machining, saw-cutting, laser cutting, grinding, milling, casting, molding, chemically etching or treating, or other known methods, and the like. In some such embodiments, the structure of the first tubular member 26 is formed by cutting and/or removing portions of the tube to form apertures 44.

In some embodiments, the apertures 44 can completely penetrate the first tubular member 26 such that there is communication between the lumen 40 and the exterior of the first tubular member 26 through the apertures 44. In some embodiments, the apertures 44 may only partially extend into the structure of the first tubular member 26, either on the interior or exterior surface thereof. Some other embodiments may include combinations of both complete and partial apertures 44 through the structure of the first tubular member 26. The shape and size of the apertures 44 can vary, for example, to achieve the desired characteristics. For example, the shape of apertures 44 can vary to include essentially any appropriate shape, such as squared, round, rectangular, pill-shaped, oval, polygonal, elongated, irregular, or the like, and may include rounded or squared edges, and can be variable in length and width, and the like.

Additionally, the spacing, arrangement, and/or orientation of the apertures 44, or in some embodiments, the spacing, arrangement, and/or orientation of the associated rings, spines or beams that may be formed due to the apertures 44, can be varied to achieve the desired characteristics. For example, the number or density of the apertures 44 along the length of the first tubular member 26, or a portion thereof, may vary, depending upon the desired characteristics. For example, the number, size, shape, or proximity of apertures 44 to one another near one region of the first tubular member 26 may be high, while the number, size, or proximity of slots to one another near another region of the first tubular member 26, may be relatively low, or vice versa. For example, in the embodiment shown in FIGS. 1 and 2, the distal portion 32 of the first tubular member 26 includes a plurality of apertures 44, while the proximal portion 28 of the first tubular member 26 does not include any apertures 44. As such, the distal portion 32 can have a greater degree of lateral flexibility relative to the proximal portion 28. Furthermore, the number, size, shape, or proximity of apertures 44 can vary within the distal portion 32 to achieve desired characteristics. For example, the number, size, shape, or proximity of apertures 44 within the distal portion 32 may be varied such that the first tubular member 26 and/or shaft 12 become more laterally flexible in the distal direction along the distal portion 28. For example, the size and density of the apertures 44 may increase in a distal direction along the first tubular member 26 and/or shaft 12, such that more lateral flexibility can be achieved in the distal direction.

As suggested above, the apertures 44 may be formed such that one or more rings interconnected by one or more spines or beams are formed in the first tubular member 26. Such rings 49 and spines or beams 50 (FIG. 1) could include portions of the tubular member 26 that remain after the apertures 44 are formed in the body of the tubular member 26. Such connected rings and/or spines or beams may act to maintain a relatively high degree of tortional stiffness, while maintaining a desired level of lateral flexibility. In some embodiments, some adjacent apertures 44 can be formed such that they include portions that overlap with each other about the circumference of the tube. In other embodiments, some adjacent apertures 44 can be disposed such that they do not necessarily overlap with each other, but are disposed in a pattern that provides the desired degree of lateral flexibility. Additionally, the apertures 44 can be arranged along the length of, or about the circumference of, the first tubular member 26 to achieve desired properties. For example, the apertures 44 can be arranged in a symmetrical pattern, such as being disposed essentially equally on opposite sides about the circumference of the first tubular member 26, or equally spaced along the length of the first tubular member, or can be arranged in an increasing or decreasing density pattern, or can be arranged in a non-symmetric or irregular pattern.

It should be understood that changes in the arrangement, number, and configuration of apertures 44 may vary without departing from the scope of the invention. Some additional examples of arrangements of cuts or slots formed in a tubular body are disclosed in U.S. Pat. No. 6,428,489 and in Published U.S. patent application Ser. No. 09/746,738 (Pub. No. US 2002/0013540), both of which are incorporated herein by reference. Also, some additional examples of arrangements of cuts or slots formed in a tubular body for use in a medical device are disclosed in a U.S. patent application Ser. No. 10/375,493 (Pub. No. US 2004/0167437), which is also incorporated herein by reference.

In addition to, or as an alternative to the structure of the first tubular member 26, the materials selected for first tubular member 26 may be chosen so that it has the desired characteristics. For example, first tubular member 26 may be formed of materials having a desired modulus of elasticity. The first tubular member 26 may be formed of any materials suitable for use, dependent upon the desired properties of the shaft 12. Some examples of suitable materials include metals, metal alloys, polymers, or the like, or combinations or mixtures thereof. Some examples of suitable metals and metal alloys include stainless steel, such as 304V, 304L, and 316L stainless steel; alloys including nickel-titanium alloy such as linear elastic or superelastic (i.e. pseudoelastic) nitinol; nickel-chromium alloy; nickel-chromium-iron alloy; cobalt alloy; tungsten or tungsten alloys; MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); hastelloy; monel 400; inconel 625; or the like; or other suitable material, or combinations or alloys thereof. In some embodiments, it is desirable to use metals, or metal alloys that are suitable for metal joining techniques such as welding, soldering, brazing, crimping, friction fitting, adhesive bonding, etc. Additionally, in some embodiments, the first tubular member 26 may be made of or include, be coated, plated, or clad with a radiopaque or MRI imaging material to facilitate radiographic visualization or MRI imaging.

The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL). In some embodiments, nitinol alloys can include in the range of about 50 to about 60 weight percent nickel, with the remainder being essentially titanium. It should be understood, however, that in other embodiment, the range of weight percent nickel and titanium, and or other trace elements may vary from these ranges. Within the family of commercially available nitinol alloys, are categories designated as “superelastic” (i.e. pseudoelastic) and “linear elastic” which, although similar in chemistry, exhibits distinct and useful mechanical properties.

In some embodiments, a superelastic alloy, for example a superelastic nitinol can be used to achieve desired properties. Such alloys typically display a substantial “superelastic plateau” or “flag region” in its stress/strain curve. Such alloys can be desirable in some embodiments because a suitable superelastic alloy will provide a reinforcing member 26 that is exhibits some enhanced ability, relative to some other non-superelastic materials, of substantially recovering its shape without significant plastic deformation, upon the application and release of stress, for example, during placement of the catheter in the body.

In some other embodiments, a linear elastic alloy, for example a linear elastic nitinol can be used to achieve desired properties. For example, in some embodiments, certain linear elastic nitinol alloys can be generated by the application of cold work, directional stress, and heat treatment, such that the material fabricated does not display a substantial “superelastic plateau” or “flag region” in its stress/strain curve. Instead, in such embodiments, as recoverable strain increases, the stress continues to increase in a somewhat linear relationship until plastic deformation begins. In some embodiments, the linear elastic nickel-titanium alloy is an alloy that does not show any martensite/austenite phase changes that are detectable by DSC and DMTA analysis over a large temperature range. For example, in some embodiments, there are no martensite/austenite phase changes detectable by DSC and DMTA analysis in the range of about −60° C. to about 120° C. The mechanical bending properties of such material are therefore generally inert to the effect of temperature over a broad range of temperature. In some particular embodiments, the mechanical properties of the alloy at ambient or room temperature are substantially the same as the mechanical properties at body temperature. In some embodiments, the use of the linear elastic nickel-titanium alloy allows the reinforcing member to exhibit superior “pushability” around tortuous anatomy. One example of a suitable nickel-titanium alloy exhibiting at least some linear elastic properties is FHP-NT alloy commercially available from Furukawa Techno Material Co. of Kanagawa, Japan. Additionally, some examples of suitable nickel-titanium alloy exhibiting at least some linear elastic properties include those disclosed in U.S. Pat. Nos. 5,238,004 and 6,508,803, which are incorporated herein by reference.

In some embodiments, the first tubular member 26 can be formed of a shape-memory material, for example a shape memory alloy such as a shape memory nitinol. In such embodiments, the shape memory effect can be used in the deployment or use of the introducer sheath 10, for example in causing the first tubular member 26 to move from a first insertion configuration to a second use configuration to effect the shape of the shaft 12, or, for example, for the first tubular member 26 to “remember” its desired shape after deformation to another shape.

For example, in some embodiments, the first tubular member 26 can include or be made of a shape memory alloy that is martensite at room temperature, and has a final austenite transition temperature (Af) somewhere in the temperature range between room temperature and body temperature. For example, in some such embodiments, the shape memory alloy has a final austenite transition temperature in the range of about 25° C. and about 37° C. (e.g. body temperature). In some such embodiments, it may be desirable that the final austenite transition temperature be at least slightly below body temperature, to ensure final transition at body temperature. This feature allows the shaft 12, including the first tubular member 26, to be inserted into the body of a patient with the first tubular member 26 in a martensitic state, and the first tubular member 26 can assume its preformed, austenitic shape when exposed to the higher body temperature within the anatomy, or at the target site, and as such effect the shape of the shaft 12. In this embodiment, deployment of the shaft 12 including the first tubular member 26 can be achieved by a shape memory effect—as the material warms, it undergoes a transition from martensite to austenite form, causing transformation of the first tubular member 26 from the first configuration to the second configuration, and thus at least partially transforming the shaft 12 from a first configuration to a second configuration.

In other example embodiments, the first tubular member 26 can include or be made of a shape-memory alloy that could have a transition temperature M_{d} (wherein M_{d} is the highest temperature to strain-induced martensite) that is in the range of body temperature (e.g. 37° C.) or greater, below which the alloy retains sufficient stress-induced martensitic property to allow placement of the shaft 12, including the first tubular member 26 at or above its final austenite transition temperature (Af). In other words, this allows the shaft 12, including the first tubular member 26 in its preformed austenitic state, to be inserted and/or navigated in the anatomy, where the first tubular member 26 may be exposed to stress that may promote portions thereof to undergo stress-induced martensitic (SIM) transformation. Thereafter, the first tubular member 26 may recover its preformed, austenitic shape when released from the stress of insertion, at a temperature that may be substantially above the final austenite transition temperature without significant plastic, or otherwise permanent deformation. Additionally, in some such embodiments, the first tubular member 26 can be restrained, for example, by a delivery device, such as an insertion and/or dilation device, in a stress-induced martensitic (SIM) state, and recover or partially recover its preformed, austenitic shape when released from the restraint, at a temperature that may be substantially above the final austenite transition temperature without significant plastic, or otherwise permanent deformation. In these embodiments, the final austenite temperature may be quite low, e.g., 4° C. or lower, or it may be up to room temperature or higher.

In yet other embodiments, the first tubular member 26 can include or be made of a shape memory alloy that is martensite at body temperature, and has a final austenite transition temperature (Af) somewhere in the temperature range above body temperature. This feature allows the shaft 12 including the first tubular member 26 to be navigated in a martensitic state, and maintain a martensitic state until exposed to a temperature higher than body temperature. The first tubular member 26 can then be heated to the necessary temperature above body temperature to make the transformation from martensite to austenite using an external heating means or mechanism. Such mechanisms may include the injection of heated fluid through the sheath, or other device, the use of electrical or other energy to heat the first tubular member 26, or other such techniques. In some such embodiments, the shape memory alloy has a final austenite transition temperature in the range of about 37° C. to about 45° C. It may be desirable that the final austenite transition temperature be at least slightly above body temperature, to ensure there is not final transition at body temperature. Some examples or Nitinol cylindrical tubes having desired transition temperatures, as noted above, can be prepared according to known methods.

As noted above, the first tubular member 26 may also be formed of or include polymer materials. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, poly(L-lactide) (PLLA), poly(D,L-lactide) (PLA), polyglycolide (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), polyethylene oxide (PEO), polydioxanone (PDS), polycaprolactone (PCL), polyhydroxylbutyrate (PHBT), poly(phosphazene), polyD,L-lactide-co-caprolactone) (PLA/PCL), poly(glycolide-co-caprolactone) (PGA/PCL), polyanhydrides (PAN), poly(ortho esters), poly(phoshate ester), poly(amino acid), polyacrylate, polyacrylamid, poly(hydroxyethyl methacrylate), polyurethane, polysiloxane and their copolymers, or mixtures or combinations thereof.

The second tubular member 24 can extend from a point within the distal portion 20 to a point within the proximal portion 16 of the shaft 12. The length of the second tubular member 24 can vary, depending upon, for example, the length of the shaft 12, the desired characteristics and functions of the sheath 10, and other such parameters. In some embodiments, the second tubular member 24 can extend substantially the entire length of the shaft 12, for example, from a point adjacent the proximal end 18 to a point adjacent the distal end 22. In yet other embodiments, the second tubular member 24 may extend proximally and/or distally beyond the first tubular member 26. As an example, the length of the second tubular member 24 can be about 5 centimeters or more, in the range of about 5 to about 100 cm, in the range of about 12 to 100 cm, or in the range of about 20 to about 100 cm.

In some embodiments, the second tubular member 24 can include a proximal portion 33 and a distal portion 35, which can be any proximal or distal sections of the second tubular member 24, but in some cases can be defined with regard to the placement of the portions of the first tubular member 26 along the length of the second tubular member. For example, in some embodiments, the distal portion 35 can be any portion of the second tubular member 24 that is within the distal portion 32 of the first tubular member 26, while the proximal portion 35 can be any portion of the second tubular member 24 that is disposed the proximal portion 28 of the first tubular member 26. In some embodiments, the distal portion 35 can have a length of about 5 cm or greater, or in the range of about 5 to about 100 cm, or in the range of about 10 to about 100 cm, or in the range of about 20 to about 100 cm. The proximal portion 35 can make up the remainder of the length of the second tubular member 24, and in some embodiments, can have a length of about 2 cm or greater, or in the range to about 2 to about 40 cm, or in the range of about 4 to about 20 cm.

As indicated above, the second tubular member 24 can define the lumen 15. The lumen 15 can be adapted and/or configured to facilitate, for example, insertion of other medical devices (e.g., guide wires, guide catheters, balloon catheters, etc.) there through, and/or to facilitate injection of fluids (e.g., radiopaque dye, saline, drugs, inflation fluid, etc.) there through. For example, the second tubular member 24 can be an inner liner disposed within the lumen 40 of the first tubular member 26 that defines the lumen 15, which can be a fluid tight pathway along at least a portion of the length of the shaft 12. For example, the second tubular member 24 can seal off and/or act as a barrier that closes the apertures 44 such that there is no fluid communication to the lumen 15 through the apertures 44. In embodiments where the second tubular member 24 may be an outer tubular member disposed about the first tubular member 26, the second tubular member 24 may still seal off and/or act as a barrier that closes the apertures 44 such that there is no fluid communication to the lumen 15 through the apertures 44. In some embodiments, the fluid tight pathway may be defined along substantially the entire length of the shaft 12. The size of the lumen 15 can vary, depending upon the desired characteristics and intended use. In some embodiments, the second tubular member 24 can have an inner diameter, defining the lumen 15, that is in the range of about 0.005 to about 0.5 inches in size, and in some embodiments, in the range of about 0.01 to about 0.3 inches in size, and in some embodiments, in the range of about 0.05 to about 0.26 inches in size. Additionally, in some embodiments, the second tubular member 24 can have an outer diameter that is in the range of about 0.005 to about 0.75 inches in size, and in some embodiments, in the range of about 0.01 to about 0.30 inches in size, and in some embodiments, in the range of about 0.05 to about 0.26 inches in size. It should be understood however, that these dimensions are provided by way of example embodiments only, and that in other embodiments, the size of the inner and outer diameter of the second tubular member 24 can vary greatly from the dimensions given, depending upon the desired characteristics and function of the device.

The second tubular member 24 may be one or more layers. In the illustrative embodiment, the second tubular member 24 may include a single layer of material, but should be understood that more or fewer layers can be used depending upon the desired characteristics of the device.

The second tubular member 24, or the layers thereof, may be made of any suitable material and by any suitable process, the materials and processes varying with the particular application. Examples of some suitable materials include, but are not limited to, polymers, metals, metal alloys, or composites or combinations thereof. Some examples of some suitable polymers can include, but are not limited to, polytetrafluoroethylene (PTFE), ethylene tetrafluoroethylene (ETFE), fluorinated ethylene propylene (FEP), high density polyethylene (HDPE), or any of the other suitable materials including any of those listed herein.

The second tubular member 24 may include a lubricious polymer such as HDPE or PTFE, for example, or a copolymer of tetrafluoroethylene with perfluoroalkyl vinyl ether (PFA) (more specifically, perfluoropropyl vinyl ether or perfluoromethyl vinyl ether), or the like. Alternatively, the second tubular member 24 may be a flexible polymer such as polyether block amide or polyether-ester elastomer. Additionally, in some embodiments, the polymer material of the second tubular member 24 can be blended with a liquid crystal polymer (LCP). For example, in some embodiments, the mixture can contain up to about 5% LCP. This has been found in some embodiments to enhance torqueability.

Additionally, as suggested above, in some embodiments, the second tubular member 24 may include or be made of metal or metal alloys. Some examples of suitable metals and metal alloys can include stainless steel, such as 304V, 304L, and 316L stainless steel; nickel-titanium alloy such as a superelastic (i.e. pseudoelastic) or linear elastic nitinol; nickel-chromium alloy; nickel-chromium-iron alloy; cobalt alloy; tungsten or tungsten alloys; tantalum or tantalum alloys, gold or gold alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); or the like; or other suitable metals, or combinations or alloys thereof. In some embodiments, it is desirable to use metals, or metal alloys that are suitable for metal joining techniques such as welding, soldering, brazing, crimping, friction fitting, adhesive bonding, etc., with the first tubular member 26, and/or with other portions of the sheath 10.

The second tubular member 24 may have a uniform stiffness, or may vary in stiffness along its length. For example, a gradual reduction in stiffness from the proximal end to the distal end thereof may be achieved, depending upon the desired characteristics. The gradual reduction in stiffness may be continuous or may be stepped, and may be achieved, for example, by varying the structure, such as the size or thickness thereof, or for example, by varying the materials used. Such variability in characteristics and materials can be achieved, for example, by using techniques such as ILC, or by fusing together separate tubular segments.

The second tubular member 24 can be formed by any suitable method or technique. For example in some embodiments, the second tubular member 24 can be formed separately, and thereafter the first and second tubular members 26/24 can be connected or attached by suitable techniques, such as friction fitting, mechanically fitting, bonding, welding (e.g., resistance, Rf, or laser welding, or the like), soldering, brazing, adhesive bonding, crimping, or the use of a connector member or material, or the like, or combinations thereof.

In some embodiments, the second tubular member 24, or other portions of the shaft 12, can define one or more additional lumens depending upon the desired characteristics and function of the introducer sheath 10, and such additional lumens can be shaped, size, adapted and/or configured the same as or different from lumen 15, depending upon the desired characteristic and functions.

Additionally, although depicted as including generally round cross-sectional shapes, it can be appreciated that the first and/or second tubular members 26/24, and or the shaft 12, can include other cross-sectional shapes or combinations of shapes without departing from the spirit of the invention. For example, the cross-sectional shapes of these structures, or portions thereof, may be oval, rectangular, square, triangular, polygonal, or a combination thereof, or the like, or any other suitable shape, depending upon the desired characteristics.

Additionally, the first and/or second tubular members 26/24, or both, or other structures or portions of the sheath 10, may be made of, include, and/or impregnated with a radiopaque material to facilitate radiographic visualization. Radiopaque materials are understood to be materials capable of producing a relatively bright image on a fluoroscopy screen or another imaging technique during a medical procedure. This relatively bright image aids the user of the introducer sheath 10 in determining its location. Some examples of radiopaque materials can include, but are not limited to, gold, platinum, palladium, tantalum, tungsten alloy, polymer material loaded with radiopaque filler, and the like. Likewise, in some embodiments, the first and/or second tubular members 26/24, or both may be made of, include, and/or impregnated with a material that may aid in MRI imaging. Some materials that exhibit these characteristics include, for example, tungsten, Elgiloy, MP35N, nitinol, and the like, and others. Those skilled in the art will recognize that these materials can vary widely without departing from the spirit of the invention.

It should also be understood that in some embodiments, a degree of MRI compatibility can be imparted into sheath 10. For example, to enhance compatibility with Magnetic Resonance Imaging (MRI) machines, it may be desirable to construct portions of the first tubular member 26, the second tubular member 24, or other portions of the sheath 10, are made in a manner, or use materials that would impart, a degree of MRI compatibility. For example, the lengths of relatively conductive structures within the sheath 10 may be limited to lengths that would not generate undue heat due to resonance waves created in such structures when under the influence of an MRI field generated by an MRI machine. Alternatively, or additionally, portions, or the entire sheath 10 may be made of a material that does not substantially distort the image and create substantial artifacts (artifacts are gaps in the image). Certain ferromagnetic materials, for example, may not be suitable because they may create artifacts in an MRI image. Additionally, all or portions of the catheter may also be made from a material that the MRI machine can image, as described above. Some materials that exhibit these characteristics include, for example, tungsten, Elgiloy, MP35N, nitinol, and the like, and others.

As indicated above, the manifold and/or hub 14 can be connected to the proximal end 18 of the elongate shaft 12, and include a lumen and/or other structure to provide access and/or fluid communication to 15 lumen within the shaft 12, and/or to facilitate the insertion of and/or connection of other medical devices (e.g., guidewire, catheter, syringe, Y-adapter, etc.) within and/or to the shaft 12. The manifold and/or hub 14 may include a hub portion 17 and a strain relief portion 19. The manifold and/or hub 14 may also include one or more valve or valve assemblies, as is generally known. Some examples of hubs including a valve assembly are disclosed in U.S. Pat. No. 6,322,541, which is incorporated herein by reference.

The manifold 14 may be secured to the shaft 10 second tubular member 24 and/or the first tubular member 26 at the proximal end 18 of the shaft 12 using any suitable technique, for example, by adhesive, friction fitting, mechanically fitting, chemically bonding, thermally bonding, heat shrink materials, molding, casting, welding (e.g., resistance or laser welding), soldering, brazing, the use of an outer sleeve or polymer layer to bond or connect the components, or the like, or combinations thereof. In some embodiments, the distal end of the manifold 14 can be cast, molded or shaped onto the proximal end 16 of the shaft 12 such that is connected to the proximal end 18, and can also act as a connector between the second tubular member 24 and/or the first tubular member 26. For example, the manifold may be made of a polymeric material, such as a polycarbonate material, or the like, that could be molded or cast onto the proximal end 16 of the shaft 12.

A lubricious, a hydrophilic, a protective, or other type of coating may be applied over portions or the entire shaft 12. Hydrophobic coatings such as fluoropolymers provide a dry lubricity which improves catheter handling and device exchanges. Lubricious coatings can aid in insertion and steerability. Suitable lubricious polymers are well known in the art and may include silicone and the like, hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

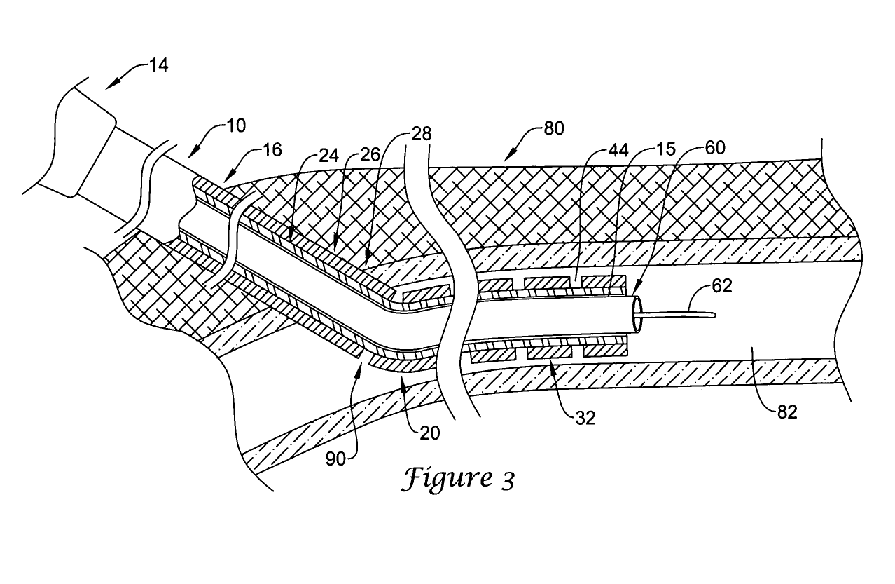

Refer now to FIG. 3, which shows an introducer sheath 10 disposed within a portion of the anatomy of a patient. As can be appreciated, the introducer sheath 10 can provide a pathway through the skin and/or other tissue 80 adjacent a vessel 82 into the vessel 82 to facilitate passage of one or more other device, such as a catheter 60, guidewire 62, or the like, or any of a broad variety of devices, fluids, medicaments, or the like, into the and/or out of the vessel, as desired.

The introducer sheath 10 can be positioned within and/or in communication with the interior of the vessel 82 using any of a broad variety of percutaneous insertion techniques generally known for inserting an introducer sheath into a vessel of a patient. For example, the use of a thin hollow needle, an insertion wire, and a dilator assembly may be used. For example, a thin metal insertion wire can be inserted percutaneously into the vessel using a thin walled hollow puncture needle, and the needle then removed to leave the insertion wire within the anatomy. A dilator can be inserted over the insertion wire and into the vessel, and the sheath 10 can be disposed on and/or advanced over the dilator for insertion into the vessel as desired.

As can be appreciated, it may be desirable that the sheath 10 include a degree of lateral flexibility, particularly within its distal portion 20, such that the sheath 10 can be adapted to enter the vessel 82 at a desired angle, and may bend or otherwise move laterally, but resist kinking. It may also be desirable that the sheath 10 include a relatively high level of pushability and torqueability, particularly within its proximal portion 16, but to a certain extent also within its distal portion 20, such that the sheath 10 can be advanced through and into the anatomy as desired. As indicated above, such characteristics may be achieved, for example, by providing the sheath 10 with an elongate tubular member, such as the first member 26, including a distal portion 32 with apertures 44 defined therein, and a proximal portion 28 not including such apertures. Such an arrangement may provide the proximal portion 16 of the sheath 12 with a desired level of pushability, torqueability, and/or stiffness, and may also provide the distal portion 20 of the sheath with a desired level of lateral flexibility relative to the proximal portion, but still include a good degree of pushability, torqueability, and/or stiffness due to the tubular structure including apertures in the wall thereof.

In some embodiments, the lengths of the proximal and distal portions 16/20 (or 28/32) may adapted or configured such that the distal portion 20, including greater flexibility characteristics, begins and/or is present and/or is positioned at a location along the length of the shaft 12 such that when the sheath 10 is used intracorporally, the distal portion 20 is present and/or corresponds with a particular portion of the anatomy that requires the shaft 12 to bend or flex relatively aggressively during use. For example, in the embodiment shown in FIG. 3, it can be appreciated that the proximal portion 16 extends along a first angle relative to the vessel 82 such that the shaft 12 can extend into the vessel 82. However, the distal portion 20, or at least a portion thereof, extends within the vessel at a different angle that may be substantially parallel with the vessel. As such, a bend region 90 can occur within the shaft 10 during use. In at least some embodiments, it may be desirable that the bend region 90 occurs within the distal portion 16, which includes apertures 44 and is more laterally flexible and better able to achieve the curve or bend. As such, in at least some embodiments, the proximal portion 16 (or 28) can have a length that is configured to extend from a point outside of the anatomy of the patient to a point adjacent to or within the vessel, and the distal portion 20 (or 32) begins at a point proximal to or within the bend region 90. As such, the bend region 90 would occur within the distal portion 16 (or 28).

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the instant specification. It should be understood that this disclosure is, in many respects, only illustrative. Changes may be made in details, particularly in matters of shape, size, and arrangement of steps without exceeding the scope of the invention. The scope of the invention is, of course, defined in the language in which the appended claims are expressed.

What is claimed is:

1. An introducer sheath, comprising:
an elongate metallic tubular member including a tubular wall defining a lumen and including a proximal portion and a distal portion, the distal portion including a plurality of apertures defined in the tubular wall, and the proximal portion is free of apertures defined in the tubular wall, the distal portion being more flexible than the proximal portion;
a second tubular member disposed on or within the elongate metallic tubular member, the second tubular member defining a fluid tight pathway through the lumen; and
a hub attached to the proximal portion of the elongate metallic tubular member and in fluid communication with the fluid tight pathway.

2. The introducer sheath of claim 1, wherein the elongate metallic tubular member includes a length, and the proximal portion extends along in the range of about 2% to about 90% of the length of the tubular member.

3. The introducer sheath of claim 1, wherein the elongate metallic tubular member includes a length, and the proximal portion extends along in the range of about 20% to about 80% of the length of the tubular member.

4. The introducer sheath of claim 1, wherein the proximal portion has a length in the range of 2 cm or greater, and the distal portion has a length in the range of 10 to about 100 cm.

5. The introducer sheath of claim 1, wherein at least some of the plurality of apertures extend through the tubular wall.

6. The introducer sheath of claim 1, wherein the elongate metallic tubular member extends along a longitudinal axis, and the apertures are elongated slots including a length and a width, the length defining a major axis of the apertures, and the major axis is disposed substantially normally to the longitudinal axis of the tubular member.

7. The introducer sheath of claim 1, wherein the distal portion of the elongate metallic tubular member has a length, and the second tubular member extends within the lumen along at least the length of the distal portion.

8. The introducer sheath of claim 1, wherein the elongate metallic tubular member has a length, and the second tubular member extends within the lumen along the length of the tubular member.

9. The introducer sheath of claim 1, wherein the elongate metallic tubular member defines an inner surface, and the second tubular member is a liner disposed on the inner surface.

10. The introducer sheath of claim 1, wherein the second tubular member extends into and is attached to the hub.

11. The introducer sheath of claim 1, wherein the elongate metallic tubular member comprises a nickel-titanium alloy.

12. The introducer sheath of claim 1, wherein the elongate metallic tubular member comprises a super elastic nickel-titanium alloy.

13. The introducer sheath of claim 1, wherein the elongate metallic tubular member comprises a linear elastic nickel-titanium alloy.

14. An introducer sheath, comprising:
an elongated tubular shaft having a proximal end and a distal end, the shaft including;
an metallic outer tubular member including a tubular wall defining an inner surface and an outer surface and defining a lumen, tubular member including a distal portion defining a plurality of apertures defined through the wall to increase the lateral flexibility of the distal portion, and a proximal portion that is free of apertures defined through the tubular wall such that the proximal portion is less laterally flexible than the distal portion; and
an inner polymer tubular member disposed within the lumen and attached to the inner surface of the tubular wall, the inner polymer member extending within the lumen and defining a fluid tight pathway through the shaft; and
a hub attached to the proximal end of the shaft and in fluid communication with the fluid tight pathway.

15. The introducer sheath of claim 14, wherein the metallic outer tubular member includes a length, and the proximal portion extends along in the range of about 2% to about 90% of the length of the tubular member.

16. The introducer sheath of claim 14, wherein the metallic outer tubular member includes a length, and the proximal portion extends along in the range of about 20% to about 80% of the length of the tubular member.

17. The introducer sheath of claim 14, wherein the elongate metallic tubular member comprises a nickel-titanium alloy.

18. The introducer sheath of claim 14, wherein the elongate metallic tubular member comprises a super elastic nickel-titanium alloy.

19. The introducer sheath of claim 14, wherein the elongate metallic tubular member comprises a linear elastic nickel-titanium alloy.

20. A method of manufacturing an introducer sheath, the method comprising:
providing an elongate metallic tubular member including a tubular wall defining a lumen and including a proximal portion and a distal portion, the distal portion including a plurality of apertures defined in the tubular wall, and the proximal portion being free of apertures defined in the tubular wall, the distal portion being more flexible than the proximal portion;
disposing an inner liner within the lumen of the tubular member to define a fluid tight pathway through the lumen; and
attaching a hub to the proximal portion of the tubular member, the hub being in fluid communication with the fluid tight pathway.
